(19) United States
(12) Patent Application Publication
(10) Pub. No.: US 2026/0335381 A1
(43) Pub. Date: Oct. 29, 2026

(54) MICROBIOME COMPOSITION OF FERMENTATION CULTURE SUPERNATANT OF LACTIPLANTIBACILLUS PLANTARUM STRAIN KM2 HAVING ANTI-INFLAMMATORY EFFECT

(71) Applicant: KOOKMINBIO, CORP., Jeonju-si, Jeollabuk-do (KR)

(72) Inventor: Moon-Hee SUNG, Seoul (KR)

(73) Assignee: KOOKMINBIO, CORP., Jeonju-si, Jeollabuk-do (KR)

(21) Appl. No.: 19/122,257

(22) PCT Filed: Oct. 18, 2023
(86) PCT No.: PCT/KR2023/016089

§ 371 Date: Apr. 18, 2025

(30) Foreign Application Priority Data
Oct. 18, 2022 (KR) 10-2022-0134158
Oct. 17, 2023 (KR) 10-2023-0138331

(51) Int. Cl. A61K 35/747; A61K 9/00; A61P 29/00

(52) U.S. Cl. CPC A61K 35/747; A61K 9/0053; A61P 29/00

(57) ABSTRACT

The present invention relates to a microbiome composition of a fermentation culture supernatant of Lactiplantibacillus plantarum strain KM2 having an anti-inflammatory effect, wherein it has been confirmed that the fermentation culture supernatant of the strain exhibits anti-inflammatory activity, such as suppressing nitric oxide activity and controlling the expression of inflammatory cytokines, and thus, the fermentation culture supernatant can be useful as a composition for preventing, treating, or ameliorating inflammatory diseases.

### TECHNICAL FIELD

The present disclosure relates to a microbiome composition of a fermentation culture supernatant of a Lactiplantibacillus plantarum KM2 strain having an anti-inflammatory efficacy.

### BACKGROUND ART

Inflammation is a normal, protective defense mechanism in vivo that occurs locally in response to tissue damage caused by physical attack, harmful chemicals, microbial infection, or irritants among metabolic products in the body. Such inflammation is triggered by a variety of chemical mediators produced by damaged tissues and migrating cells, and these chemical mediators are known to be very diverse depending on the type of inflammatory processes. In normal cases, the body neutralizes or eliminates pathogenic factors through an inflammatory response and regenerates damaged tissues to restore normal structures and functions.

However, when these inflammatory responses take place abnormally, it may progress to diseases such as chronic inflammation, and when inflammation is inappropriately triggered by harmless substances such as pollen or autoimmune reactions such as asthma or rheumatoid arthritis, the defense response itself may rather damage tissues, causing various diseases.

Currently, steroidal and non-steroidal drugs are used as the most commonly used treatment for the prevention or treatment of inflammatory diseases, but most of them have side effects, such that efforts are actively being made to develop drugs for inflammatory diseases that overcome the shortcomings above.

### DISCLOSURE OF THE INVENTION

### Technical Goals

An object of the present disclosure is to provide a pharmaceutical composition for preventing or treating an inflammatory disease.

Another object of the present disclosure is to provide a health functional food composition for preventing or ameliorating an inflammatory disease.

Another object of the present disclosure is to provide a method of preventing or treating an inflammatory disease.

### Technical Solutions

To achieve the above objects, the present disclosure provides a pharmaceutical composition for preventing or treating an inflammatory disease, including a fermentation culture supernatant of a Lactiplantibacillus plantarum strain, a concentrate thereof, a dried product thereof, a fermentation metabolite thereof, or a mixture thereof as an active ingredient.

In addition, the present disclosure provides a health functional food composition for preventing or ameliorating an inflammatory disease, including a fermentation culture supernatant of the strain, a concentrate thereof, a dried product thereof, a fermentation metabolite thereof, or a mixture thereof as an active ingredient.

In addition, the present disclosure provides a method of preventing or treating an inflammatory disease, including treating the pharmaceutical composition according to claim 1 to a patient group having an inflammatory disease with a decreased number of strains of one or more selected from the group consisting of Weizmannia coagulans, Clostridium perfringens, Clostridium botulinum, Clostridium sporogenes, Clostridium baratii, and Clostridium butyricum compared to a normal group; or a patient group having an inflammatory disease with an increased number of Bifidobacterium pseudolongum strains compared to a normal group.

### Advantageous Effects

According to the present disclosure, it was found that a fermentation culture supernatant of a Lactiplantibacillus plantarum KM2 strain exhibits an anti-inflammatory activity, such as inhibition of nitric oxide activity and regulation of expression of inflammatory cytokines, such that the fermentation culture supernatant may be usefully utilized as a composition for preventing, treating, or ameliorating an inflammatory disease.

### BRIEF DESCRIPTION OF DRAWINGS

FIG. 1 shows results of analyzing a cytotoxicity of a fermentation culture supernatant (hereinafter referred to as sample) of a Lactiplantibacillus plantarum KM2 strain in macrophages.

FIG. 2 shows a result of analyzing an effect of a sample on nitric oxide (hereinafter referred to as NO) production in macrophages.

FIG. 3 shows a result of analyzing an effect of a sample on prostaglandin E2 (hereinafter referred to as PGE2) production in macrophages.

FIG. 4 shows results of analyzing an effect of a sample on expression of inducible nitric oxide synthase (iNOS) and cyclooxygenase-2 (COX-2) in macrophages.

FIG. 5 shows results of analyzing an effect of a sample on production of pro-inflammatory and anti-inflammatory cytokines in macrophages.

FIG. 6 shows a result of analyzing an effect of a sample on trans-epithelial electrical resistance (TEER) in intestinal epithelial cells.

FIG. 7 shows a result of analyzing an effect of a sample on paracellular permeability in intestinal epithelial cells.

FIG. 8 shows results of analyzing an effect of a sample on expression of intestinal junction proteins in macrophages.

FIG. 9 shows results of analyzing an effect of a sample on a body weight and a disease activity index score in an animal model.

FIG. 10 shows results of analyzing an effect of a sample on a colon length in an animal model.

FIG. 11 shows results of analyzing an effect of a sample on inflammatory cytokine production in an animal model.

FIG. 12 shows results of analyzing an effect of a sample on intestinal tissues in an animal model.

FIG. 13 shows results of analyzing an effect of a sample on intestinal microorganisms in an animal model.

It is indicated that all drawings have statistical differences among treated groups represented by each different letter (e.g. a, b, etc.) at a 95% confidence level. For example, if there are group 1 represented as a, group 2 as b, and group 3 as ab, there is a statistical difference at a 95% level between groups 1 and 3 and between groups 1 and 2; and there is no statistical difference between groups 2 and 3.

### BEST MODE FOR CARRYING OUT THE INVENTION

Hereinafter, the present disclosure will be described in more detail.

The present disclosure provides a pharmaceutical composition for preventing or treating an inflammatory disease, including a fermentation culture supernatant of a Lactiplantibacillus plantarum strain, a concentrate thereof, a dried product thereof, a fermentation metabolite thereof, or a mixture thereof as an active ingredient.

The strain may be a Lactiplantibacillus plantarum KM2 strain deposited under deposit number KCTC 14637BP.

The inflammatory disease may be one or more selected from the group consisting of ulcerative colitis, ulcerative duodenitis, Crohn's disease, irritable bowel syndrome, intestinal Behcet's disease, hemorrhagic rectal ulcer, pouchitis, enteritis, ischemic colitis, acne, dermatitis with extra-intestinal manifestations, atopic dermatitis, allergic dermatitis, seborrheic dermatitis, papular urticaria, eczema, asthma, conjunctivitis, periodontitis, rhinitis, otitis media, iritis, pharyngitis, tonsillitis, pneumonia, pancreatitis, gastritis, hemorrhoids, gout, ankylosing spondylitis, lupus, fibromyalgia, psoriasis, rheumatoid arthritis, osteoarthritis, osteoporosis, hepatitis, cystitis, nephritis, Sjogren's syndrome, and multiple sclerosis, but is not limited thereto.

The pharmaceutical composition may further include killed cells or spores of the Lactiplantibacillus plantarum KM2 strain.

In addition, the pharmaceutical composition may regulate one or more intestinal microorganisms selected from the group consisting of Weizmannia coagulans, Clostridium perfringens, Clostridium botulinum, Clostridium sporogenes, Clostridium baratii, Clostridium butyricum, and Bifidobacterium pseudolongum.

In addition, the pharmaceutical composition may inhibit production or expression of one or more selected from the group consisting of prostaglandin E2, inducible nitric oxide synthase (iNOS), cyclooxygenase-2 (COX-2), interleukin-1β (IL-1β), tumor necrosis factor-α (TNF-α), and interleukin-6 (IL-6), or promote production or expression of one or more selected from the group consisting of ZO-1, occludin, claudin-1, and interleukin-10 (IL-10), but is not limited thereto.

The pharmaceutical composition of the present disclosure may be prepared in the form of a unit dose by formulation using a pharmaceutically acceptable carrier or prepared by encapsulating into a multi-capacity container, in accordance with a method that may be easily carried out by a person with ordinary skill in the art to which the disclosure pertains.

The pharmaceutically acceptable carriers are commonly used in preparation, including, but are not limited to, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil. The pharmaceutical composition of the present disclosure may further include lubricants, humectants, sweeteners, flavoring agents, emulsifiers, suspensions, and preservatives, in addition to the above ingredients.

In the present disclosure, the content of the additives included in the pharmaceutical composition is not particularly limited and may be appropriately adjusted within a content range used in the conventional preparation.

The pharmaceutical composition may be formulated in the form of one or more external skin agents selected from the group consisting of, but are not limited to, injectable formulations such as aqueous solutions, suspensions, and emulsions, pills, capsules, granules, tablets, creams, gels, patches, sprays, ointments, plasters, lotions, liniments, pastas, and cataplasmas.

The pharmaceutical composition of the present disclosure may include a pharmaceutically acceptable carrier and diluent that are additionally present for formulation. The pharmaceutically acceptable carriers and diluents include, but are not limited to, excipients such as starch, sugars, and mannitol, fillers and bulking agents such as calcium phosphate, cellulose derivatives such as carboxymethylcellulose and hydroxypropylcellulose, binders such as gelatin, alginate, and polyvinyl pyrrolidone, lubricants such as talc, calcium stearate, hydrogenated castor oil, and polyethylene glycol, disintegrating agents such as povidone and crospovidone, and surfactants such as polysorbate, cetyl alcohol, and glycerol. The pharmaceutically acceptable carriers and diluents may be biologically and physiologically friendly to a subject. Examples of diluents may include, but are not limited to, brine, water-soluble buffers, solvents, and/or dispersion media.

The pharmaceutical composition of the present disclosure may be administered orally or parenterally (e.g., applied intravenously, subcutaneously, intraperitoneally, or topically) depending on the desired method. When administered orally, it may be formulated as tablets, troches, lozenges, water-soluble suspensions, oil-based suspensions, powder preparation, granules, emulsions, hard capsules, soft capsules, syrups, and elixirs. When administered parenterally, it may be formulated as injection solutions, suppositories, powders for respiratory inhalation, aerosols for sprays, ointments, powders for application, oils, and creams.

The dosage range of the pharmaceutical composition of the present disclosure may vary depending on the patient's condition, weight, age, sex, health status, dietary constitution specificity, nature of the preparation, severity of a disease, administration time for composition, method of administration, duration or interval of administration, excretion rate, and drug form, and may be appropriately selected by a person skilled in the art. For example, it may range from about 0.1 to 10,000 mg/kg, but is not limited thereby, and it may be administered once or several times a day.

The pharmaceutical composition may be administered orally or parenterally (e.g., applied intravenously, subcutaneously, intraperitoneally, or topically) depending on the desired method. The pharmaceutically effective amount and effective dosage of the pharmaceutical composition of the present disclosure may vary by the preparation method of the pharmaceutical composition, type of administration, administration time, and administration route, and a person with ordinary skill in the art may easily determine and prescribe the effective dosage for the desired treatment. The administration of the pharmaceutical composition of the present disclosure may be administered once a day or in several divided doses.

In addition, the present disclosure provides a health functional food composition for preventing or ameliorating an inflammatory disease, including a fermentation culture supernatant of a Lactiplantibacillus plantarum strain, a concentrate thereof, a dried product thereof, a fermentation metabolite thereof, or a mixture thereof as an active ingredient.

The strain may be a Lactiplantibacillus plantarum KM2 strain deposited under deposit number KCTC 14637BP.

The present disclosure may be used generally as a food product that is conventionally used.

The food composition of the present disclosure may be used as a health functional food. The term “health functional food” as used herein refers to a food manufactured and processed using raw materials or ingredients that have useful functionality for the human body in accordance with the Health Functional Food Act, and the term “functionality” as used herein refers to consumption for the purpose of deriving useful effects for health purposes such as adjusting nutrients or physiological actions on the structure and function of the human body.

The health functional food composition may include conventional food additives, and the suitability as the “food additive” is determined by the standards and criteria associated with corresponding items according to the general rules and general test methods of Korean Food Additives Codex approved by the Ministry of Food and Drug Safety, unless otherwise stipulated.

The items listed in the “Korean Food Additives Codex” may include, for example, chemically synthesized compounds such as ketones, glycine, potassium citrate, nicotinic acid, and cinnamic acid, natural additives such as persimmon color, licorice extracts, crystallized cellulose, kaoliang color, and guar gum, and mixed preparations such as sodium L-glutamate preparations, noodle-added alkali agents, preservative agents, and tar color agents.

The food composition of the present disclosure may be manufactured and processed in the form of tablets, capsules, powder, granules, liquids, and pills. For example, hard capsule preparations among health functional foods in the form of capsules may be prepared by mixing and filling the composition according to the present disclosure in conventional hard capsules along with additives such as excipients, and the soft capsule preparations may be manufactured by mixing the composition according to the present disclosure with the additives such as excipients and then filling the same in capsule bases such as gelatin. The soft capsule preparations may include, if necessary, plasticizers such as glycerin or sorbitol, colorants, and preservatives.

The definition of terms for the excipient, binder, disintegrant, lubricant, flavor enhancer, and flavoring agent is described in documents known in the art and includes those having the same or similar functions. The type of food is not particularly limited and includes all health functional foods in the ordinary sense.

The term “prevention” as used herein refers to any action of suppressing or delaying inflammatory diseases by administering the composition according to the present disclosure. The term “treatment” as used herein refers to any action of ameliorating or changing the condition of inflammatory diseases better by administering the composition according to the present disclosure. The term “amelioration” as used herein refers to any action of making the bad condition of inflammatory diseases better by administering the composition according to the present disclosure.

In addition, the present disclosure provides a method of preventing or treating an inflammatory disease, including treating the pharmaceutical composition to a patient group having an inflammatory disease with a decreased number of strains of one or more selected from the group consisting of Weizmannia coagulans, Clostridium perfringens, Clostridium botulinum, Clostridium sporogenes, Clostridium baratii, and Clostridium butyricum compared to a normal group; or a patient group having an inflammatory disease with an increased number of Bifidobacterium pseudolongum strains compared to a normal group.

### MODES FOR CARRYING OUT THE INVENTION

Hereinafter, the present disclosure will be described in more detail through examples to help understanding of the present disclosure. However, examples below are merely intended to illustrate the present disclosure, and the scope of the present disclosure is not limited to the following examples. Examples of the present disclosure are provided to more completely explain the present disclosure to those skilled in the art.

### [Experimental Example 1] Preparation of Samples

To prepare a fermentation culture supernatant of a KM2 strain (Lactiplantibacillus plantarum KM2), which is a sample of this experiment, a KM2 stock strain stored at −70 °C was activated followed by primary seed culture in a test tube and flask, 2% (v/v) thereof was inoculated by 20 L working volume in a 50 L fermenter, and secondary seed culture was performed for 6 hours. This culture was performed in a 500 L fermenter by 2% (v/v) inoculation in a 350 L working volume for 12 hours, and glucose was additionally fed once at 6 hours after the culture. After completion of the culture, the cell slurry was removed by primary centrifugation in a disk centrifuge at 7,200 rpm and 2 L/min, and the supernatant was secondarily centrifuged in a tubular centrifuge at 15,000 rpm and 1.5 L/min to remove the cell cake and recover the supernatant. The recovered supernatant was filtered through a 0.2 μm sterilizing filter to obtain the fermentation culture supernatant of the KM2 strain from which the bacterial cells were finally eliminated. The sterilized fermentation culture supernatant was added with 3% (w/v) trehalose as an excipient and then freeze-dried for 96 to 120 hours to obtain a powdered fermentation culture supernatant.

### [Experimental Example 2] Cell Culture

Raw 264.7 cells, a mouse-derived macrophage cell line, were obtained from the Korean Cell Line Bank (KCLB). Cells were subcultured once every two days in an incubator in the presence of 5% CO₂ at 37 °C using Dulbecco's modified Eagle's medium (DMEM) supplemented with 10% fetal bovine serum (FBS) and 1% penicillin/streptomycin (hereinafter referred to as P/S).

IPEC-J2 cells, which are porcine intestinal epithelial cells, were obtained from the German Collection of Microorganisms and Cell Cultures GmbH (DSMZ). Cells were subcultured once every two days in an incubator in the presence of 5% CO₂ at 37 °C using Dulbecco's Modified Eagle's Medium-Nutrient-Mixture F-12 (DMEM/F-12) medium supplemented with 10% FBS and 1% P/S.

### [Experimental Example 3] Preparation of Animal Models

C57BL/6 mice were purchased from Orient Bio. Healthy animals were used in the experiment by checking the number of mice, observing general symptoms, measuring body weight, and checking the test results provided by the animal supplier. All animals were checked for abnormalities and given a 7-day acclimatization (adaptation) period to adapt to the animal room environment. During the acclimatization period, all animals were observed for general symptoms once a day, and on the final day of acclimatization, weight changes were checked to evaluate the health status of the animals. The animals were divided into four groups, with nine animals per group, to ensure that the average body weight of each experimental group was equal. Individual marks were made on the animals' tails using a five-colored permanent marker, and individual identification cards were attached to breeding boxes. Animal models were raised under conditions of temperature of 20 to 26 °C, relative humidity of 30 to 70%, and a 12-hours of a day light/dark cycle (9:00 AM-9:00 PM) and supplied with food and drinking water. The animals were freely fed solid feed for laboratory animals (Lab DFiet #5053 PMI Nutrition International, U.S.A). Animal experiments were conducted with the approval of the Institutional Animal Care and Use Committee of the Korea Food Industry Cluster (IACUC-22-014).

### [Experimental Example 4] Statistical Analysis

All experimental results were expressed as mean±standard error (mean±SEM), and all statistical analyses were performed using the SAS program (release 9.4 SAS Institute Inc., Cary, NC, USA). Significant differences (p<0.05) between experimental groups were analyzed using one-way ANOVA and Duncan's multiple range test.

### [Example 1] Analysis of Physiological Activity of Samples (In Vitro)

### 1-1. Cytotoxicity Assay

To identify the cytotoxicity of the sample in macrophages and intestinal epithelial cells, the concentration of the sample affecting cell viability was determined based on the decrease in absorbance according to the sample treatment concentration compared to the control group (sample untreated group; control) using the EZ-Cytox kit (DAEIL lab, Korea). Raw 264.7 and IPEC-J2 cells were seeded at a density of 1×10⁴ cells/well in 96-well plates, respectively and cultured for 24 hours in an incubator in the presence of 5% CO₂ at 37 °C, and then samples were treated at various concentrations, followed by culture for 24 hours. After that, the culture medium was removed, 90 μL of DMEM and 10 μL of EZ-Cytox reagent were mixed and dispensed, and a reaction was carried out for 1 hour in an incubator in the presence of 5% CO₂ at 37 °C in a dark room. Afterwards, the absorbance was measured at 450 nm using a microplate reader, and the cell viability was measured using the following Equation 1.

Cell viability=(Absorbance of a control group)/(Absorbance of a sample treated group)×100 [Equation 1]

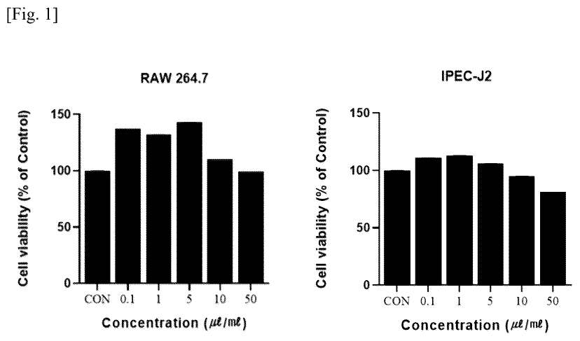

As a result, as shown in FIG. 1, in the case of Raw 264.7 cells, no significant cytotoxicity was observed in the group treated with 0.1 to 50 μl/ml sample, and in the case of IPEC-J2 cells, no significant cytotoxicity was observed in the group treated with 0.1 to 10 μl/ml sample. Based on the above results, an experiment was performed on the physiological activity of the sample in the concentration band where no cytotoxicity was observed.

### 1-2. Analysis of NO Production

To determine the effect of the sample on nitric oxide (NO) production, NO activity was measured using a nitric oxide (NO) assay kit (Promega Corp, USA, Cat. #G2930). Raw 264.7 cells were seeded at a density of 2.5×10⁵ cells/well in a 24-well plate and cultured for 24 hours in an incubator in the presence of 5% CO₂ at 37 °C, and then culture was followed for 24 hours by diluting 1 μg/ml of lipopolysaccharides (LPS, Sigma-Aldrich, USA) (derived from Escherichia coli 055:B5) and the samples at various concentrations using DMEM medium supplemented with 1% FBS and 1% P/S. Afterwards, the amount of NO produced during cell culture was measured. For the amount of NO produced, 50 μL of cell culture medium and 100 μL of Griess reagent (Sigma-Aldrich Co.) were mixed and reacted at room temperature for 10 minutes according to the kit protocol. The absorbance of the colored reaction solution was measured at 520 nm using a microplate reader. Nitrite concentration was calculated using the standard curve.

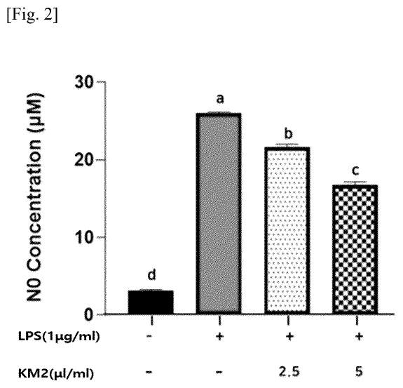

As a result, as shown in FIG. 2, NO production significantly increased in the LPS only treated group compared to the control group (LPS and sample untreated group), and NO production significantly decreased in a concentration-dependent manner in the sample treated group compared to the LPS only treated group.

### 1-3. Analysis of an Inhibitory Activity on PGE2 Production

To identify an effect of the sample on prostaglandin E2 (PGE2) production, Raw 264.7 cells were seeded at a density of 2.5×10⁵ cells/well in a 24-well plate and cultured for 24 hours in an incubator in the presence of 5% CO₂ at 37 °C, and then culture was followed for 24 hours by diluting 1 μg/ml of lipopolysaccharides (LPS, Sigma-Aldrich, USA) (derived from Escherichia coli 055:B5) and the samples at various concentrations using DMEM medium supplemented with 1% FBS and 1% P/S. Afterwards, each cell culture medium was taken and centrifuged at 13,000 rpm for 10 minutes, and the PGE2 content of the supernatant obtained through centrifugation was measured. All samples were stored frozen at −20 °C until quantification, PGE2 was quantified using a mouse enzyme-linked immunosorbent assay (ELISA) kit (Cat. #KGE004B, R&D Systems Inc., Minneapolis, MN, USA), and the R2 value of the standard curve for the standard was 0.99 or higher.

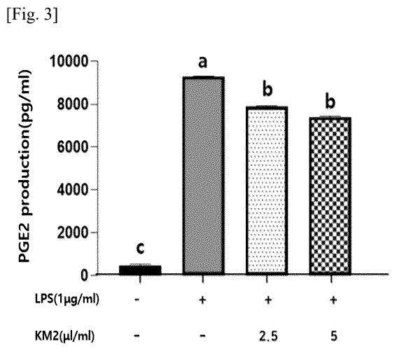

As a result, as shown in FIG. 3, PGE2 production significantly increased in the LPS only treated group compared to the control group (LPS and sample untreated group), and PGE2 production significantly decreased in the sample treated group compared to the LPS only treated group.

### 1-4. Analysis of an Inhibitory Activity on iNOS and COX-2 Expression

To determine the effect of the sample on the expression of inducible nitric oxide synthase (iNOS) and cyclooxygenase-2 (COX-2), qPCR was performed. To isolate total RNA from Raw 264.7 cells, a Trizol reagent kit (Invitrogen, USA) was used, and RNA was precipitated using 100% isopropanol, followed by washing with 75% ethanol. The extracted RNA was dissolved in distilled water from which nuclease had been removed, and its concentration was measured using a nanospectrophotometer. cDNA was synthesized using Maxime RT pre-Mix (Oligo dt 15 Primer, iNtRON Biotechnology, Korea) according to the method provided by the manufacturer. To analyze the gene expression of iNOS (NCBI Gene ID: 396859) and COX-2 (NCBI Gene ID: 808504), KAPA SYBR fast qPCR Kit (KAPA biosystems, USA) was used. qPCR conditions were set as 1 cycle at 95 °C for 5 minutes, 35 cycles at 96 °C for 20 seconds, 60 °C for 20 seconds, and 72 °C for 20 seconds, and 1 cycle at 72 °C for 5 minutes. Data analysis was performed using Light Cycler 96 software provided by the manufacturer (Roche Applied Science). Quantitative results were expressed by comparison with reference MRNA (GAPDH) using the 2^{-ΔΔCT} method (Livak & Schmittgen, 2001).

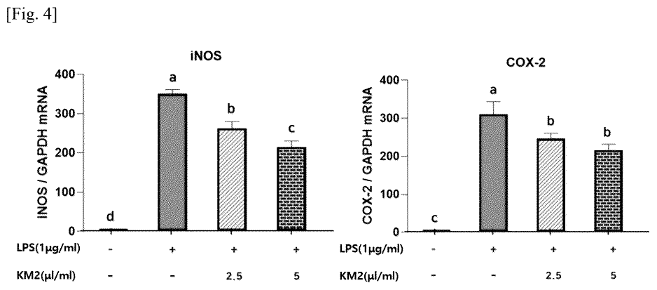

As a result, as shown in FIG. 4, the expression of iNOS and COX-2 significantly increased in the LPS only treated group compared to the control group (LPS and sample untreated group), and the expression of iNOS and COX-2 significantly decreased in a concentration-dependent manner in the sample treated group compared to the LPS only treated group.

### 1-5. Analysis of Production of Proinflammatory and Anti-Inflammatory Cytokines

To identify an effect of the sample on the production of pro-inflammatory cytokines and anti-inflammatory cytokines, the production of these cytokines was measured using ELISA kits for IL-1β (NCBI Gene ID: 16176) (Interleukin-1β, Cat. #MLB00C), TNF-α (NCBI Gene ID: 21926) (tumor necrosis factor-α, Cat. #MTA00B), IL-6 (NCBI Gene ID: 16193) (Interleukin-6, Cat. #DY406-05), and IL-10 (NCBI Gene ID: 16153) (Interleukin-10, Cat. #M1000B-1). The experiment was conducted using Raw 264.7 cell culture supernatant according to the kit protocol. The absorbance of the colored reaction solution was measured at 450 nm using a microplate reader. The concentration of each cytokine was calculated using the standard curve.

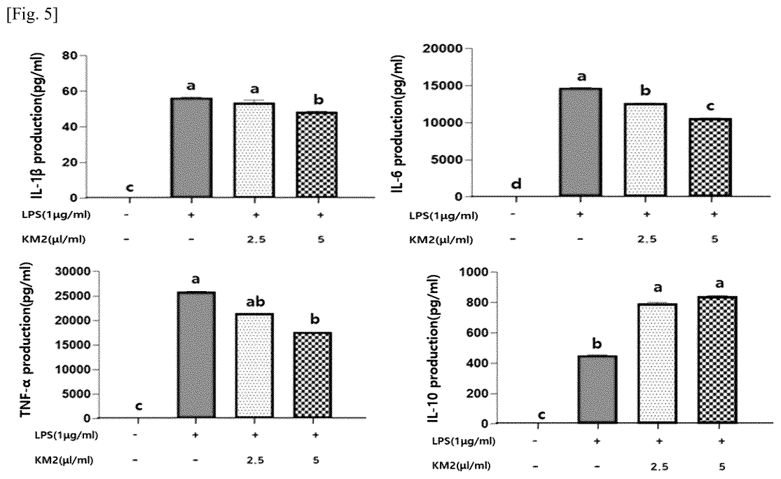

As a result, as shown in FIG. 5, the production of IL-1β, IL-6, and TNF-α significantly increased in the LPS only treated group compared to the control group (LPS and sample untreated group), and the production of the three cytokines significantly decreased in the sample treated group compared to the LPS only treated group. In addition, IL-10 production significantly increased in the LPS only treated group and the sample treated group, and the increase in IL-10 production was more significant in the sample treated group compared to the LPS only treated group.

### 1-6. Analysis of Trans-Epithelial Electrical Resistance (TEER)

To identify an effect of a sample on trans-epithelial electrical resistance (TEER), TEER of IPEC-J2 cell monolayers cultured on 0.4 μm transwell membranes (Corning, NY, USA) was analyzed using a Millicell-ERS-2 instrument (Millipore, MA, USA). IPEC-J2 cells were dispensed into 24 transwells at a density of 2×10⁵ cells/well and cultured for 24 hours at 37 °C in the presence of 5% CO₂. Afterwards, the upper wells were replaced with 500 μL of the concentration-specific sample, the lower wells were replaced with 1.5 mL of medium, and the plates were cultured for an additional 24 hours (37 °C, 5% CO₂, and 95% humidity). Afterwards, 1 μg/mL lipopolysaccharides (LPS, Sigma-Aldrich, USA) (derived from Escherichia coli 055:B5) dissolved in Hank's Balanced Salt Solution (HBSS) was treated, and each TEER value was measured at regular intervals immediately after sample treatment using a Miller-ERS instrument (Millipore, MA, USA). The TEER value was calculated using the following Equation 2.

TEER=Resistance (Ohm)×Filter area (cm²)=Ω×cm² [Equation 2]

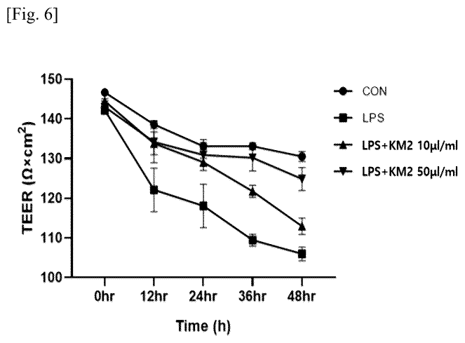

As a result, as shown in FIG. 6, the TEER value decreased over time in all experimental groups, and the decrease in the TEER value was most significant in the LPS only treated group, revealing that the decrease in the TEER value was suppressed in the sample treated group compared to the LPS only treated group.

### 1-7. Paracellular Permeability Assay

To identify an effect of the sample on paracellular permeability, IPEC-J2 cells were treated with 1 μg/mL LPS for 48 hours, and then paracellular permeability was measured using 4 kDa FITC-dextran (Sigma-Aldrich, USA). For the cells treated with the LPS, FITC-dextran was dissolved in HBSS and dispensed into the upper well at a concentration of 1 mg/ml followed by culture for 4 hours, and then the paracellular permeability was measured using a Tecan Reader (excitation, 492 nm; emission, 520 nm, Tecan Group Ltd., Switzerland).

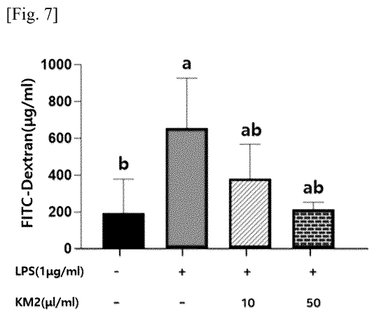

As a result, as shown in FIG. 7, the flux of FITC-dextran significantly increased in the LPS only treated group compared to the control group (LPS and sample untreated group), and the flux of FITC-dextran significantly decreased in a concentration-dependent manner in the sample treated group compared to the LPS only treated group.

### 1-8. Analysis of Expression of Intestinal Junction Proteins

To identify an effect of a sample on the expression of intestinal junction proteins, IPEC-J2 cells cultured with LPS for 48 hours were washed once with Dulbecco's phosphate-buffered saline (DPBS), PRO-PREP reagent was dispensed at 100~150 μl per well, and the cells were harvested into a tube using a scraper. The harvested cells were left on ice for 30 minutes and then centrifuged for 5 minutes at 13,000 rpm and 4 °C to obtain the supernatant. Afterwards, the supernatant was quantified for protein using the Bradford assay, and 50 μg of protein was separated using 8% SDS-PAGE and transferred to a polyvinylidene fluoride (PVDF) membrane. Afterwards, blocking was performed for 1 hour using blocking buffer (4% nonfat dry milk, 10 mM Tris, 100 mM NaCl, and 0.1% Tween 20, pH 7.5), followed by washing three times for 15 minutes each with 1% Tween 20-PBS. ZO-1 (NCBI Gene ID: 100736682), occludin (NCBI Gene ID: 397236), claudin-1 (NCBI Gene ID: 100625166), and β-actin antibodies (primary antibodies) were treated at room temperature for 3 hours, and then washing was performed with 1% Tween 20-PBS. Afterwards, secondary antibodies were treated for 1 to 2 hours followed by washing, and the protein expression levels of zonula occludens-1 (ZO-1), occludin, and claudin-1 were measured with an enhanced chemiluminenscence (ECL) kit using a LAS 4000 instrument for detection.

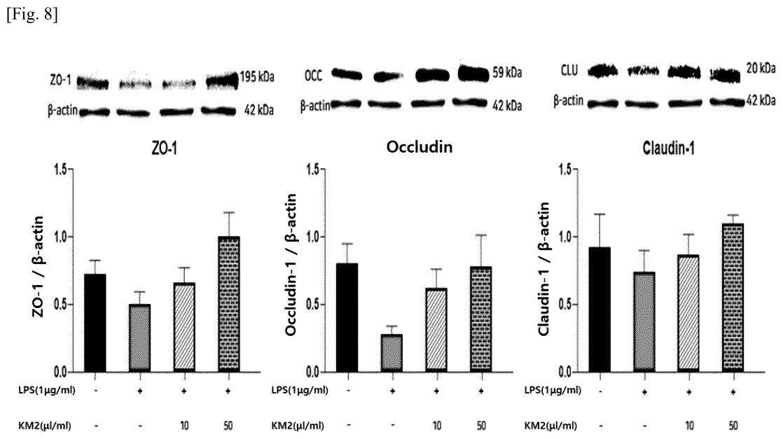

As a result, as shown in FIG. 8, the protein expression of ZO-1, occludin, and claudin-1 decreased in the LPS only treated group compared to the control group (LPS and sample untreated group), and the expression of the three types of proteins increased in a concentration-dependent manner in the sample treated group compared to the LPS only treated group.

### [Example 2] Analysis of Physiological Activity of Samples (In Vivo)

### 2-1. Fabrication of an Enteritis-Induced Animal Model

To create an animal model induced with enteritis, 3% dextran sulfate sodium (DSS) salt was provided as drinking water to the animal model of Experimental Example 3 to induce enteritis. As a positive control, 50 mg/kg of 5-aminosalicyclic acid (5-ASA) was used, and the sample and 5-ASA were orally administered into the stomach of the animal model once daily for 5 weeks from the start of 3% DSS intake using a disposable syringe (1 ml) attached to an oral administration sonde. The experimental group was specifically set up as follows.
1) Normal group (CON)
2) Negative control group (DSS): 3% DSS intake
3) Positive control group (DSS+ASA): 3% DSS intake+50 mg/kg 5-ASA administered orally
4) Sample treated group (DSS+KM2): 3% DSS intake+2 g/kg sample administered orally

### 2-2. Analysis of the Body Weight and Disease Activity Index Score

To identify an effect of the sample on the body weight and disease activity index (DAI) score, the body weight of the animal model was measured once a day for 5 weeks (DSS intake period), and the stool condition (stool form and color of bloody stool) was checked using a blood stool kit to measure the disease activity index score (stool form, color of bloody stool, and weight loss).

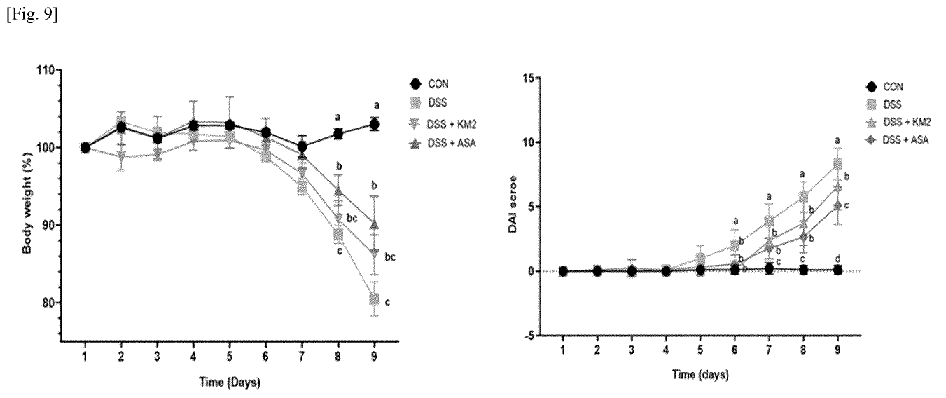

As a result, as shown in FIG. 9, the body weight decreased in the negative control group (DSS) compared to the control group (CON), and the body weight increased in the positive control group (DSS+ASA) and sample treated group (DSS+KM2) compared to the negative control group. In addition, from the fifth day of induction of enteritis (DSS intake), weight loss and bloody stool significantly increased in the negative control group, positive control group, and sample treated group to lead to an increase in the disease activity index score compared to the control group, and the disease activity index score in the positive control group and sample treated group decreased compared to the negative control group.

### 2-3. Analysis of a Colon Length

To identify an effect of the sample on a colon length, animal models were fasted for 24 hours, sacrificed, and dissected to collect blood from the heart, followed by observation of appearance, abdominal cavity, and thoracic cavity macroscopically. Afterwards, the colon was removed, and its length was measured.

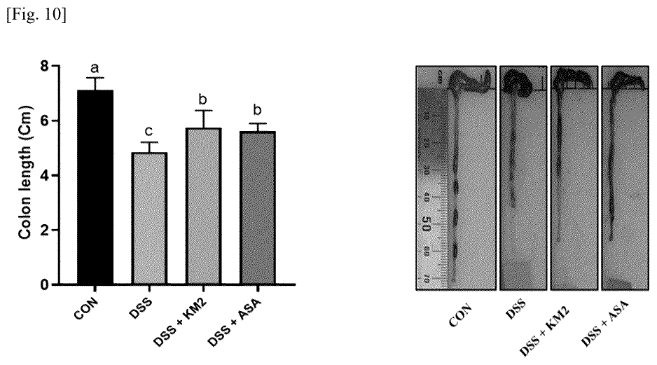

As a result, as shown in FIG. 10, the colon length significantly decreased in the negative control group (DSS) compared to the control group (CON), and the colon length increased in the positive control group (DSS+ASA) and sample treated group (DSS+KM2) compared to the negative control group.

### 2-4. Blood Biochemical Analysis (Analysis of Inflammatory Cytokine Production)

To identify an effect of the sample on the production of inflammatory cytokines, the animal models were sacrificed, and then blood was collected from the heart and immediately centrifuged to separate serum which was then stored in an ultra-low temperature freezer at −80 °C until analysis. Afterwards, the cytokine content was measured using a spectrophotometer and analysis kit.

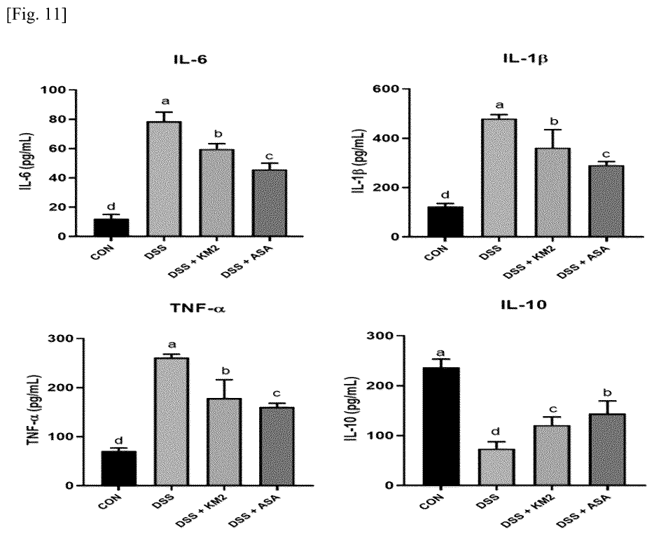

As a result, as shown in FIG. 11, the production of IL-6, IL-1β, and TNF-α significantly increased in the negative control group (DSS) compared to the control group (CON), and the production of IL-10 significantly decreased. In contrast, in the positive control group (DSS+ASA) and sample treated group (DSS+KM2), IL-6, IL-1β, and TNF-α production significantly decreased, and IL-10 production significantly increased compared to the negative control group.

### 2-5. Histological Analysis

To identify an effect of the sample on intestinal tissues, the colon tissue of the animal model was fixed with 10% formaldehyde, paraffin blocks were created, and Hematoxylin & Eosin (H&E) staining was performed. After photographing the stained tissue slides, quantitative image analysis was performed. Additionally, the degree of intestinal mucus secretion was analyzed by performing alcian blue staining.

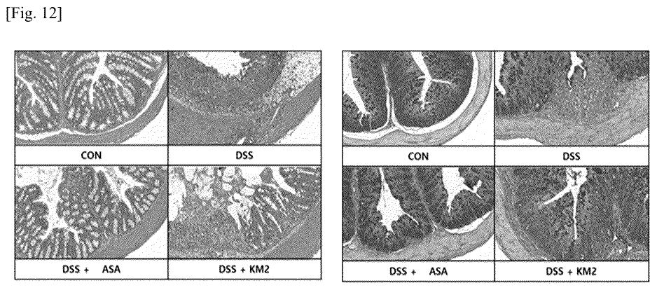

As a result, as shown in FIG. 12, the structure and size of the epithelial tissue forming the colon surface changed irregularly, and the infiltration of inflammatory cells increased in the negative control group (DSS) compared to the control group (CON). On the other hand, it was found that the symptoms appearing in the negative group were improved in the positive control group (DSS+ASA) and sample treated group (DSS+KM2) compared to the negative control group. In addition, the degree of blue staining decreased in the negative control group compared to the control group in which the mucosal layer was stained blue, whereas the degree of blue staining increased in the positive control group and sample treated group compared to the negative control group.

### 2-6. Analysis of Intestinal Microorganisms

To identify an effect of the sample on the intestinal microorganism, the animal models were sacrificed, the cecum was removed, and changes in the intestinal microorganisms were analyzed using shotgun metagenome sequencing.

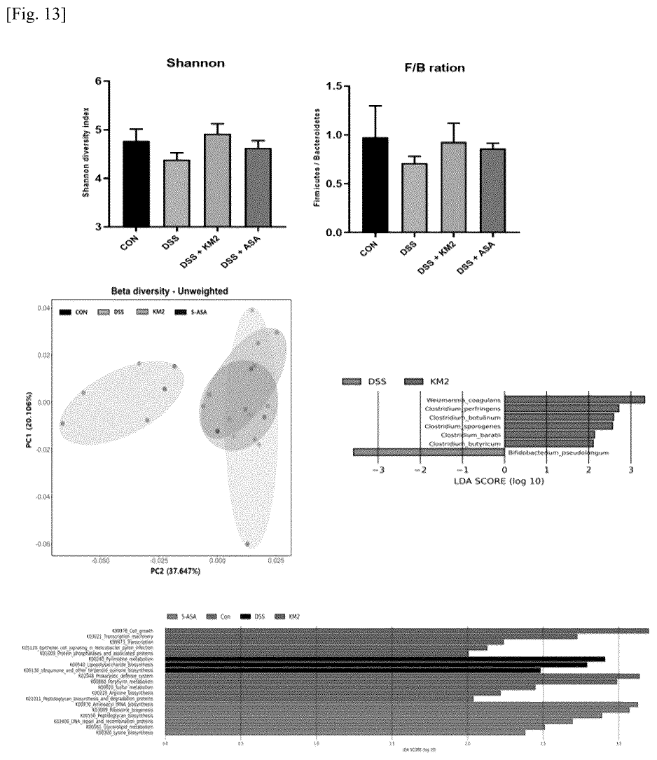

As a result, as shown in FIG. 13, in the α-diversity analysis results that allow identification of a diversity of microorganisms existing in a single sample, it was found that there was no significant difference in the Shannon index between the experimental groups. Additionally, there was no significant difference in the Firmicutes/Bacteroidetes (F/B) ratio, which is an inflammation index, between the experimental groups. The results of β-diversity analysis based on unweighted UniFrac principal coordinate analysis (PCA), which allows identification of the microbial colony pattern, showed significant differences between the experimental groups. To identify significantly different microbiomes between each experimental group, as a result of conducting linear discriminant analysis (LDA) and KEGG pathway analysis, as shown in FIG. 13, the number of Weizmannia coagulans, Clostridium perfringens, Clostridium botulinum, Clostridium sporogenes, Clostridium baratii, and Clostridium butyricum strains significantly increased in the sample treated group (DSS+KM2) compared to the negative control group (DSS). Additionally, the number of Bifidobacterium pseudolongum strains significantly increased in the negative control group compared to the sample treated group.

While a specific part of the present disclosure has been described in detail above, it is clear for those skilled in the art that this specific description is merely preferred example embodiments, and the scope of the present disclosure is not limited thereby. In other words, the substantial scope of the present disclosure is defined by the attached claims and their equivalents.

1. A pharmaceutical composition for preventing or treating an inflammatory disease, comprising a fermentation culture supernatant of a Lactiplantibacillus plantarum strain, a concentrate thereof, a dried product thereof, a fermentation metabolite thereof, or a mixture thereof as an active ingredient.

2. The pharmaceutical composition of claim 1, wherein the strain is a Lactiplantibacillus plantarum KM2 strain deposited under deposit number KCTC 14637BP.

3. The pharmaceutical composition of claim 1, wherein the inflammatory disease is one or more selected from the group consisting of ulcerative colitis, ulcerative duodenitis, Crohn's disease, irritable bowel syndrome, intestinal Behcet's disease, hemorrhagic rectal ulcer, pouchitis, enteritis, ischemic colitis, acne, dermatitis with extra-intestinal manifestations, atopic dermatitis, allergic dermatitis, seborrheic dermatitis, papular urticaria, eczema, asthma, conjunctivitis, periodontitis, rhinitis, otitis media, iritis, pharyngitis, tonsillitis, pneumonia, pancreatitis, gastritis, hemorrhoids, gout, ankylosing spondylitis, lupus, fibromyalgia, psoriasis, rheumatoid arthritis, osteoarthritis, osteoporosis, hepatitis, cystitis, nephritis, Sjogren's syndrome, and multiple sclerosis.

4. The pharmaceutical composition of claim 1, wherein the pharmaceutical composition further comprises killed cells or spores of the Lactiplantibacillus plantarum KM2 strain.

5. The pharmaceutical composition of claim 1, wherein the pharmaceutical composition regulates one or more intestinal microorganisms selected from the group consisting of Weizmannia coagulans, Clostridium perfringens, Clostridium botulinum, Clostridium sporogenes, Clostridium baratii, Clostridium butyricum, and Bifidobacterium pseudolongum.

6. A health functional food composition for preventing or ameliorating an inflammatory disease, comprising a fermentation culture supernatant of a Lactiplantibacillus plantarum strain, a concentrate thereof, a dried product thereof, a fermentation metabolite thereof, or a mixture thereof as an active ingredient.

7. The health functional food composition of claim 1, wherein the strain is a Lactiplantibacillus plantarum KM2 strain deposited under deposit number KCTC 14637BP.

8. A method of preventing or treating an inflammatory disease, comprising treating the pharmaceutical composition according to claim 1 to a patient group having an inflammatory disease with a decreased number of strains of one or more selected from the group consisting of Weizmannia coagulans, Clostridium perfringens, Clostridium botulinum, Clostridium sporogenes, Clostridium baratii, and Clostridium butyricum compared to a normal group; or a patient group having an inflammatory disease with an increased number of Bifidobacterium pseudolongum strains compared to a normal group.
